SEQUENCE LISTING (6 sequences)

SEQ ID NO: 1 - DNA, 6822 bp, Homo sapiens
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 2 - DNA, 776 bp, Human cytomegalovirus
gtagccatgctctggaagatcttcaatatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactaga

SEQ ID NO: 3 - DNA, 805 bp, Homo sapiens
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcc

SEQ ID NO: 4 - DNA, 1042 bp, Homo sapiens
ccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggtctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgttt

SEQ ID NO: 5 - DNA, 672 bp, Homo sapiens
gtgccacctggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 6 - DNA, 7268 bp, Mus musculus
gctggacgtggtcctagcggcctttgtgtccggtgcttgcctgagccccagctcggggtgattctcccggtgctggggtggtccggcatgggcttcctcagacagatacagcttttgctttggaagaactggactctgaggaaaaggcagaagattcgctttgtagtggaactcgtgtggcctttgtctttgtttttggtgttaatctggctgaggaatgccaacccactctatagtcagcatgaatgccattttcccaacaaggcgatgccttcagcaggactgttaccgtggctccaggggattttctgcaatatgaacaacccttgttttcaaaaccccacccctggagagtctcctggaactgtctcaaactataacaactccatcttggcaagagtatatcgagattttcaagaactcttcatggacaccccggaggtccagcaccttggccaggtttgggccgagctccgcaccttgtcgcagttcatggacaccctgaggactcaccctgagagatttgcaggaagaggattacaaatccgagacatcctaaaagatgaagaggcgctgaccctatttctcatgagaaacattggcctgtctgactcggttgcccatcttctggtcaactcccaagttcgtgtggagcagtttgcttatggagtcccagacttggaactgacagacattgcctgcagcgaggccctcctgcagcgcttcatcatcttcagccagcgtcggggggcacagacggtacgcgatgccctgtgtcccctctcccaggtcaccctacagtggatagaagacactctgtatgccgatgtggacttcttcaaactcttccatgtgctccccacactcctggacagcagttctcaaggaatcaacttgagattttggggaggaatattatctgatctgtcaccaagaatgcaaaagtttatccatcggccaagtgttcaagacttgctatgggtgagcagacctctcctgcagaacggtggtcctgagaccttcacacagctgatgagcatcctgtctgacctcctgtgtgggtacccagagggaggaggctcccgagtgttctcctttaactggtatgaagacaataactataaagccttcctggggattgattccacaaggaaagaccccgcctattcttacgacaaaagaacaacatccttttgtaattcattgatccagagcctggagtcaaaccctttaaccaaaatagcctggagggcggcaaagccattgctgatgggaaaaatcctctttactccagattcccctgctgctcgaaggataatgaagaatgccaactcaacttttgaagaactggatcgagttaggaagttggtaaaagcctgggaggaagtggggccccagatctggtacttctttgagaagagcacacagatgaccgtgatccgagacaccctgcagcacccaaccgtcaaagacttcataaataggcagctcggagaagaaggcattaccaccgaagccgtattaaacttcttctctaacggtccccaagagaagcaggctgatgatatgaccagctttgactggagggacatattcaacatcactgaccgattcctacgcttggctaatcaatacctggagtgtctggtcctggataagtttgaaagttatgatgatgaagtgcagctcacccaacgagccctgtctctcctggaggagaacaggttctgggccggagtggtgttccctggcatgtatccctgggccagctccttacctcctcatgtgaagtacaagattcggatggacatagatgtggtggagaagaccaataagatcaaagacaggtactgggattctggtcccagggcggatcctgtcgaagatttccggtacatctggggaggttttgcctatctacaggacatggtggagcaaggaatcgtgaagagtcagatgcaggcagagcctccaattggagtctatctccaacagatgccttatccctgctttgtggatgactccttcatgatcatcctgaatcgctgtttccctatcttcatggtgctggcgtggatctactctgtctccatgaccgttaagggcattgttttggagaaggagctgaggctgaaggagaccttgaaaaaccaaggcgtctctaatgctgtcatctggtgtacctggttcctggatagcttctccatcatggcgctgagtatcttcctcctgacgctgttcatcatgcatggaaggatcctccattacagcgatcccttcattctcttcctgttcttgttggcctttgccactgcgaccatcatgcagagcttcctgctcagtactttgttttccaaggccagccttgcagcagcctgcagtggggtcatctacttcaccctctacctaccacacgttctgtgctttgcctggcaggaccggatgacggccgacctgaagacgactgtgagcctactttcttccgtggcatttgggtttggcaccgagtacctggtccgctttgaggagcaaggcctggggctgcaatggagcaacattgggaagagtcccctggaaggggatgagttcagtttcctgctgtctatgaagatgatgcttctcgatgctgctctctacggcttgcttgcttggtatcttgaccaggttttcccaggagactatgggaccccacttccctggtacttccttctgcaggagtcctactggcttggtggtgaaggttgttcgaccagagaagaaagggctctggaaaagactgaacccttaacagaggagatggaggatccagagcacccagaaggaatgaatgactccttttttgaacgcgagcttccagggctggtgcctggtgtgtgtgtgaagaacctggtgaaggtttttgagcccagtggccggccagctgtggaccgtctgaacatcactttctatgagaaccaaatcacggcgttcctgggtcacaacggagcgggaaagaccaccaccttgtccatcctgacaggactgttgccaccgacgtcaggaactgtgctcattgggggaaaagacattgaaaccaacctggatgtagtacggcagagcctgggcatgtgtccacaacacaacatcctgtttcatcacctcacggtggctgagcacatcttgttctatgcccagctgaaagggagatcctgggaggaggcccagcttgagatggaagccatgctagaagacacgggcctccaccataagaggaatgaagaagctcaggacctttcaggtggcatgcagaggaagctgtctgttgccattgcttttgtgggagattctaaggtggtggtcctggatgagcccacctctggggtggacccctactccagacgctccatctgggacctgctcctgaagtatcgctcaggcagaaccatcatcatgtccactcaccacatggacgaggcagacctccttggggaccgcattgccatcatttctcagggaaggctctactgctctggaaccccgctcttcctcaagaactgctttggcacaggcttctacttgaccttagttcgcaagatgaaaaacatccagagccaaagaggtggctgtgagggggtctgcagctgtacatcaaagggtttctctaccaggtgtccaacccgagtcgatgagataacagaagaacaagtcctggatggagatgtgcaggagctgatggatttggtataccaccatgtcccagaggcaaagctggtggaatgcattggtcaagaacttatcttcctccttccaaacaagaatttcaagcagagagcatatgccagccttttccgagagctggaggagactctggctgacctggggctcagcagctttggaatttctgacactcccctggaagagattttcctgaaggtcacagaggatgctggagcgggctctatgtttgtaggtggcgctcagcagaaaagagaacaagccggtctccggcacccttgttcggctcccactgagaagctcaggcagtatgcccaggccccacatacctgttccccaggacaagtggatcctcccaagggccaaccttccccagagccagaggaccccggtgtcccattcaacacaggtgctcggctgattcttcaacatgtgcaggccctgctagtcaagcgattccatcacaccatccgcagccgcaaggactttgtggctcagattgttctccctgccactttcgtgttcttggctctgatgctttcaatcattgtgcctccgtttggtgaatttccagctttgactcttcatccctggatgtatgggcatcaatataccttcttcagcatggacgaacccaacaatgaacaccttgaagtactggcagatgtccttctgaacaggccaggctttggcaaccgttgtctaaaggaagagtggcttccggagtacccatgcattaatgcaacctcctggaagacgccctccgtgtccccgaacatcacccacctgttccagaagcagaaatggacagcagcccacccctctccctcctgcaagtgcagcaccagagagaagctcaccatgttgcccgagtgccccgagggtgctggagggctcccacccccacagaggacacagcgcagcactgaagtcctacaagacctcacgaacaggaacatctccgactacttggtaaaaacgtaccctgctctcataagaagcagcttaaagagcaaattctgggtcaatgaacagaggtatggaggaatttccatcggaggaaaactccctgctatccccatcagcggggaagcacttgttggctttctaagtggccttggccagatgatgaacgtgagcgggggtcctgtcaccagggaggcctccaaagaaatgttagatttcctcaaacatcttgaaaccacagacaacattaaggtatggtttaacaacaagggctggcatgccctggtcagctttctgaatgtggctcacaacgccatcttacgggccagcctgcccagggacagggaccctgaggagtatggaatcactgtcatcagccagcctctgaacctgaccaaggagcagctctcggacatcacagtactgactacttccgtggatgccgtggttgccatatgtgtgattttcgccatgtccttcgttccagccagctttgtcctttacttgatccaggagagggtgacgaaagctaaacatttgcagtttatcagtggtgtgagccccaccacctactggctgaccaacttcctctgggatattatgaattatgctgtgagtgcgggattggtggtgggcatcttcattggatttcaaaagaaagcctacacatccccagacaaccttcctgctctcgtctctctgctcatgctgtatggatgggcagtcattcctatgatgtatccagcatccttcctgtttgaagtccccagcacagcctatgtggctttgtcctgtgctaacctgttcatcggcatcaacagcagcgccatcaccttcgtcctggaattatttgagaataaccggacgctgctcaggttcaatgccatgttgaggaagttgctcattgtcttcccccacttctgcctgggcaggggcctcattgacctggcactgagccaagctgtgacagacgtctatgcccagtttggtgaggagtactctgcaaacccattccagtgggacctgattgggaagaacctggttgctatggcaatagaaggggtggtatacttccttctgaccctgctcatccaacaccattttttcctcacccggtggatcgctgagcctgctagagagcccgtttttgatgaagatgacgatgtggctgaagaaagacaaagagttatgagtgggggaaataaaaccgacatcttaaagctaaacgaactaaccaaggtttactcaggctcttccagtccagcagtagatcggttatgtgttggagttcgacctggagagtgctttggcctcctgggagtgaatggtgcaggcaaaaccaccacattcaagatgctcactggggacaccacagtgacatcgggggatgctactgtggcaggcaagagcattttaactagtatttctgatgtccatcaaaacatgggctactgtcctcagtttgatgcaatcgatgacctgctcacaggcagagaacacctttacctctatgccaggctgcggggcgtgccatcgaaggaaatcgagaaggttgccaactggggtatccagagcctgggcttgtctctctacgctgaccgcctggcaggcacctacagtggaggcaataagaggaaactctctacagccatagctctcactggctgccctcccctgctgctgctggatgagcccacgacagggatggatccccaggcacgccgcatgctgtggaacaccattgtgagcatcatcagagaagggagagctgtggttctcacctcccacagcatggaagaatgtgaagctctgtgtacgcggctggccatcatggtgaagggcacctttcagtgtctgggtaccatccaacacctcaagtacaagtttggagacggctacattgtcacaatgaaaatcaaatctccaaaggacgacttgcttcccgatttgaatcctgtggagcagttcttccagggcaacttccctggcagcgtgcagagggagagacaccacagcatgctccagttccaggtcccctcgtcctccctggccaggatcttccagctgctcatttcccacaaggacagcctgctcatcgaggagtactcagtcacccagaccacgctggaccaggtgtttgtaaacttcgctaaacagcagactgagacctatgacctccctctgcacccccgggctgctggagccagctggcaagccaagcttgaagagaaatccggacggctgcagacacaggagcctttgcccgcaggatctgaacaactggctaacggaagcaaccccacggcagcagaagataagcacaccaggagtccacagtgagctggaggagactctttcagaaagaaaccaaaattcactggctcaccggaactcctgatggcaaaactgacctatcagaatggtcacctgtttatccagaacccctgactggtggctttggccttagcgatactttcattctgagtggatctgcttttgtggagtgggaagggtatggatgtgtgggtgttatttgaggggagaaataaaaaacagtctcctgattaaaaaaaaaaaaaaaaaaaaaa

(19) United States
(12) Patent Application Publication
(10) Pub. No.: US 2009/0214478 A1
(43) Pub. Date: Aug. 27, 2009

(54) Method of treating ocular diseases by gene therapy

(76) Inventor: Alberto Auricchio, Napoli (IT)

Correspondence Address: ARENT FOX LLP, 1050 CONNECTICUT AVENUE, N.W., SUITE 400, WASHINGTON, DC, 20036, US

(21) Appl. No.: 12/071,508

(22) Filed: Feb. 21, 2008

(51) Int. Cl. A61K 35/76; C12N 15/00; A61P 27/00

(52) U.S. Cl. 424/93.2; 435/320.1

(57) ABSTRACT

A method for the treatment of diseases associated with mutations in ABCA4 gene by administering, to a subject in need thereof, an adeno-associated viral vector encoding an ABCR protein; genetic constructs and adeno-associated viral vectors for use in this method.

The present invention provides a method for the treatment of diseases associated with mutations in ABCA4 gene by administering, to a subject in need thereof, an adeno-associated viral vector encoding the ABCR (“ATP-binding cassette transporter-retinal”) protein. The invention also includes genetic constructs and adeno-associated viral vectors for use in this method.

### BACKGROUND OF THE INVENTION

Stargardt's Disease (Deutman, A.F.a.H.C.B. 2001. Macular dystrophies. St Louis, Mo., Usa: Schachat, A. P. 1210-1257 pp.) (STGD) is an autosomal recessive hereditary disease included in the group of degenerative macular diseases, which consists in progressive lost of cones in fovea of both eyes, leading to variable levels of central vision loss. At fundoscopy, the presence of yellowish flecks around the macula is often observed, a condition called fundus flavimaculatus. It usually develops in ages between 7 and 12, with an estimated prevalence of 1/10,000 individuals, which makes this disease the largest cause of inherited macular degeneration affecting the photoreceptor cells in the first and second decades of life, and correspond to 7% of all retinian dystrophies. This disease was first described as an autosomal recessive inherited disease, but there are some described cases of dominant pattern. The recessive pattern, which includes more than 90% of cases, is due to a defect at the chromosome 1q21-p13. The dominant pattern seems to be related to a change at chromosome 6, but some studies also reported the location on chromosome 12.

The gene responsible for recessive Stargardt's disease has been identified as the ABCA4 gene (Allikmets, R., et al. 1997. A photoreceptor cell-specific ATP-binding transporter gene (ABCR) is mutated in recessive Stargardt macular dystrophy. Nat Genet 15:236-246) which encodes the ABCR protein, a member of the ATP-binding cassette (ABC) transporter family. It is expressed in photoreceptors and has been localized to the rim of outer segment discs.

Other diseases associated with mutations in ABCA4 include cone-rod dystrophy (Maugeri, A., et al, 2000, “Mutations in the ABCA4 (ABCR) gene are the major cause of autosomal recessive cone-rod dystrophy” Am J Hum Genet 67:960-966.) and retinitis pigmentosa (Cremers, F. P., et al. 1998. “Autosomal recessive retinitis pigmentosa and cone-rod dystrophy caused by splice site mutations in the Stargardt's disease gene ABCR” Hum Mol Genet 7:355-362; Martinez-Mir, et al. 1998. “Retinitis pigmentosa caused by a homozygous mutation in the Stargardt disease gene ABCR” Nat Genet 18:11-12). Importantly, heterozygous ABCA4 mutations in humans (Allikmets, R. et al. 1997 “Mutation of the Stargardt disease gene (ABCR) in age-related macular degeneration” Science 277:1805-1807) have been associated with age-related macular degeneration (AMD), the most common blinding disease in the elderly (Seddon, J. M. 2001. Epidemiology of Age-Related Macular Degeneration. St Louis, Mo., USA: Schachat, A. P. 1039-1050 pp).

### DESCRIPTION OF THE INVENTION

The invention is based on the finding that the administration, preferably intraocular administration, of ABCA4-encoding adeno-associated viral vectors with AAV5 capsids results in protein localization to rod outer segments and in significant and stable morphological and functional improvement of the Abca4−/− retina. In particular it has been found that subretinal delivery of rAAV2/5-CMV-Abca4 in an animal model of STGD results in significant correction of lipofuscin levels, RPE abnormalities and retinal function.

These findings provide a valuable therapeutic approach to recessive Stargardt's disease, the most common inherited macular degeneration, as well as other diseases associated with mutations in ABCA4 such as cone-rod dystrophy, retinitis pigmentosa and age-related macular degeneration (AMD), the most common blinding disease in the elderly.

Accordingly, in a first aspect the invention is directed to a method for correcting retinal abnormalities and/or retinal function in a mammalian subject, particularly in a human individual affected by a disease associated with mutations in ABCA4 gene, said disease being preferably selected from recessive Stargardt's disease, cone-rod dystrophy, retinitis pigmentosa and age-related macular degeneration (AMD), the method of the invention comprising the steps of:
1) providing a recombinant adeno-associated viral (AAV) vector with AAV5 capsid, said vector carrying an expression cassette which contains a nucleic acid molecule encoding a functional ABCR protein, wherein said nucleic acid molecule is operably linked to regulatory control elements that direct the transcription and translation thereof;
2) transducing photoreceptor cells with said recombinant AAV vector, whereby the expression of the ABCR protein is induced in said cells.

Vectors with AAV5 capsids proved able of packaging genomes up to 9 kb, preferably from about 4.7 to 9 kb, more efficiently than other serotypes, therefore their use for delivering the ABCA4 gene according to the invention is preferred. The recombinant AAV2/5 vector, which is preferably delivered to the subretinal space resulting in production of functional ABCR protein of the appropriate molecular weight and biological activity, is particularly preferred.

By “functional ABCR protein” applicant means that the ABCR protein exhibits the function of the native protein, e.g. the protein binds ATP sufficiently in vivo to provide function to the photoreceptor cells. Preferably, the functional ABCR protein exhibits at least 80%, more preferably at least 90%, and most preferably at least 95% of the function of the native protein. Determination of functional activity can be conducted, for example, in accordance with procedures described in Sun et al., Nature Genetics 26, 242-246 (2000), hereby incorporated by reference.

For the purposes of this invention, a coding sequence of ABCA4, which is preferably selected from SEQ ID NO:1 (human) and SEQ ID NO:6 (murine), or sequences encoding the same amino acid sequence due to the degeneracy of the genetic code, is functionally linked to a promoter sequence able to regulate the expression thereof in a mammalian retinal cell, particularly in photoreceptor cells. Suitable promoters that can be used according to the invention include the CMV (SEQ ID NO:2), human RHO (SEQ ID NO:3), human ABCA4 (SEQ ID NO:4) and CBA (SEQ ID NO:5) promoters, fragments and variants thereof retaining a transcription promoter activity.

The construction of an AAV vector can be carried out following procedures and using techniques which are known to a person skilled in the art. The theory and practice for adeno-associated viral vector construction and use in therapy are illustrated in several scientific and patent publications (the following bibliography is herein incorporated by reference: Flotte T R. Adeno-associated virus-based gene therapy for inherited disorders. Pediatr Res. December 2005; 58(6):1143-7; Goncalves M A. Adeno-associated virus: from defective virus to effective vector, Virol J. May 6, 2005; 2:43; Surace E M, Auricchio A. Adeno-associated viral vectors for retinal gene transfer. Prog Retin Eye Res. November 2003; 22(6):705-19; Mandel R J, Manfredsson F P, Foust K D, Rising A, Reimsnider S, Nash K, Burger C. Recombinant adeno-associated viral vectors as therapeutic agents to treat neurological disorders. Mol Ther. March 2006; 13(3):463-83).

In a further aspect, the invention relates to a pharmaceutical composition containing an AAV vector expressing the ABCA4 coding sequence, preferably in a form suitable for ocular administration. Suitable administration forms include, but are not limited to, injectable solutions or suspensions, eye lotions and ophthalmic ointment. In a preferred embodiment, the AAV vector is administered by subretinal injection, e.g. by injection in the subretinal space, in the anterior chamber or in the retrobulbar space. Preferably the viral vectors are delivered via subretinal approach (as described in Bennicelli J, et al Mol Ther. Jan. 22, 2008; Reversal of Blindness in Animal Models of Leber Congenital Amaurosis Using Optimized AAV2-mediated Gene Transfer).

The doses of virus for use in therapy shall be determined on a case by case basis, depending on the administration route, the severity of the disease, the general conditions of the patients, and other clinical parameters. In general, suitable dosages will vary from 10⁹ to 10¹³ vg (vector genomes)/eye.

### DESCRIPTION OF THE FIGURES

FIG. 1. Genome integrity of rAAV2/5-CMV-Abca4 (A) Southern blot analysis of vector DNA isolated directly from rAAV large preps (2.5×10¹⁰ GC/lane) and separated on alkaline agarose gels. Lane 1 contains a marker DNA fragment obtained by restriction digestion from the pAAV2.1-CMV-Abca4 plasmid; lane 2 contains the same DNA fragment as in lane 1 digested with Dnase I, as control of Dnase I activity; lanes 3 and 4: genomes isolated from rAAV2/5-CMV-Abca4. Sample in lane 3 was treated with Dnase I. (B) Assessment of rAAV2/5-CMV-Abca4 genome length following in vivo delivery. (top panel) Schematic representation of the rAAV2/5-CMV-Abca4 genome with the 2 probes used for the Southern blot analysis. (middle panel) Southern blot analysis of genomic DNA from uninjected muscles (lanes 1 and 3) and an equivalent amount of genomic DNA from murine muscle injected with rAAV2/5-CMV-Abca4 (lane 2 and 4) digested with Ncol and Notl (lanes 1 and 2) or Ncol alone (lanes 3 and 4). Lanes belong to the same gel but were non-contiguous. The arrows point to the bands of the expected size. (bottom panel) Southern blot analysis with a probe specific for the PDE6B gene used as loading control. Molecular weights are indicated on the left. (C) Western blot analysis with anti-ABCA4 (top panel) or anti-α tubulin (bottom panel) antibodies of lysates from Cos cells transduced with rAAV2/5. Lane 1: retina from wild-type mouse; lane 2: samples transduced with rAAV2/5-CMV-Abca4; lane 3: samples transduced with rAAV2/5-CMV-EGFP. Anti-α tubulin was used as loading control. The amount (micrograms, μg) of protein loaded are indicated under the respective lanes.

FIG. 2. ABCA4 expression following rAAV2/5 delivery.

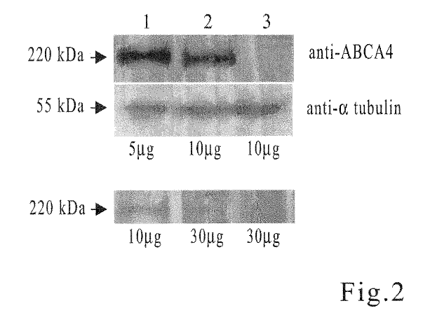

Western blot analysis with anti-ABCA4 (top panel), anti-α tubulin (middle panel) antibodies and 8-Azido-[α-³²P]-ATP labelling of ABCA4 (bottom panel) of lysates from Abca4−/− retinas transduced with rAAV2/5. Lane 1: retina from wild-type mouse; lane 2: samples transduced with rAAV2/5-CMV-Abca4; lane 3: samples transduced with rAAV2/5-CMV-EGFP. Anti-α tubulin was used as loading control. The amount (micrograms, μg) of protein loaded are indicated under the respective lanes.

FIG. 3. Morphological analysis of Abca4−/− retinas following rAAV-mediated gene transfer (A) Immunohistochemical analysis with anti-ABCA4 (Rim 3F4) antibody of retinal sections from 4 month-old Abca4+/+ mice and Abca4−/− pigmented mice injected subretinally at 1 month of age with rAAV2/5-CMV-EGFP and the controlateral eye with rAAV2/5-CMV-Abca4. RPE, retinal pigment epithelium; OS, outer segment (photoreceptors); ONL, outer nuclear layer; INL, inner nuclear layer; GCL, ganglion cell layer. Magnification 20×. (B) Electron microscopy analysis of retinal pigment epithelium from pigmented 5-month old Abca4−/− mice. Retinal pigment epithelium (RPE) from one eye injected subretinally at 1 month of age with rAAV2/5-CMV-EGFP (left) and the controlateral eye with rAAV2/5-CMV-Abca4 (right). Ch, Choroid; BrM, Bruch's membrane. White arrows indicate the irregularly shaped lipofuscin pigment granules to be distinguished from the larger oval melanosomes. Micrographs were obtained at the same magnification (6,000×). (C) Number of lipofuscin granules (left) and RPE thickness (right) in the RPE of Abca4+/+ or Abca4−/− mice injected subretinally with rAAV2/5-CMV-EGFP or rAAV2/5-CMV-Abca4 (n=2 eyes/group).

FIG. 4. Reduction of lipofuscin levels and improved recovery from photoreceptor desensitization in Abca4−/− mice injected with rAAV2/5-CMV-Abca4. (A) Effect of rAAV2/5-mediated Abca4 gene transfer on lipofuscin accumulation in the retina of Abca4−/− mice. A2E (combined A2E and iso-A2E), atRALdi-E and atRALdi-PE levels in eyecups of 4 and 6-month old albino and pigmented Abca4−/− mice, respectively, injected at post-natal day 30 in one eye with rAAV2/5-CMV-Abca4 (gray columns) and in the controlateral eye with rAAV2/5-CMV-EGFP (empty columns). Age-matched albino Balb/c and pigmented Abca4+/+ mice are represented in striped columns. Values are the average of two independent samples containing 4 eye cups each. (B) Rescue from delayed recovery from photoreceptor desensitization in Abca4−/− mice treated with rAAV2/5-CMV-Abca4. Progressive recovery after bleaching of the b-wave amplitude in 4-month old Abca4−/− mice injected subretinally with either rAAV2/5-CMV-Abca4 (red triangles, n=4 eyes) or rAAV2/5-CMV-EGFP (green squares, n=4 eyes) and in age matched wild-type Balb/c mice (black circles, n=10 eyes). Data are shown as average ± standard error. Asterisks depict statistically significant differences (P≦0.05).

### EXPERIMENTAL SECTION

Methods

Generation of the Plasmid Constructs

For the production of rAAV encoding EGFP and ABCA4, the pAAV2.1-CMV-EGFP (Auricchio, A., et al. J. M. 2001. Isolation of Highly Infectious and Pure Adeno-Associated Virus Type 2 Vectors with a Single-Step Gravity-Flow Column. Hum Gene Ther 12:71-76) and pZac2.1-CMV-Abca4 plasmids were used (CMV sequence from NC001347.3 nt 174661 to 175243). The pZac2.1-CMV-Abca4 was obtained by cloning the murine Abca4 cDNA (7,268 bp, including the coding sequence as well as some 5′ and 3′ UTR region) between the EcoRI and Sall sites in the pZac2.1 plasmid (Gao, G., et al. J. M. 2000. Purification of recombinant adeno-associated virus vectors by column chromatography and its performance in vivo. Hum Gene Ther 11:2079-2091). The Abca4 cDNA was obtained from the pBluescript SK(−)Abca4 plasmid by digestion with EcoRI and Xhol enzymes.

Animal Models and Vector Administration

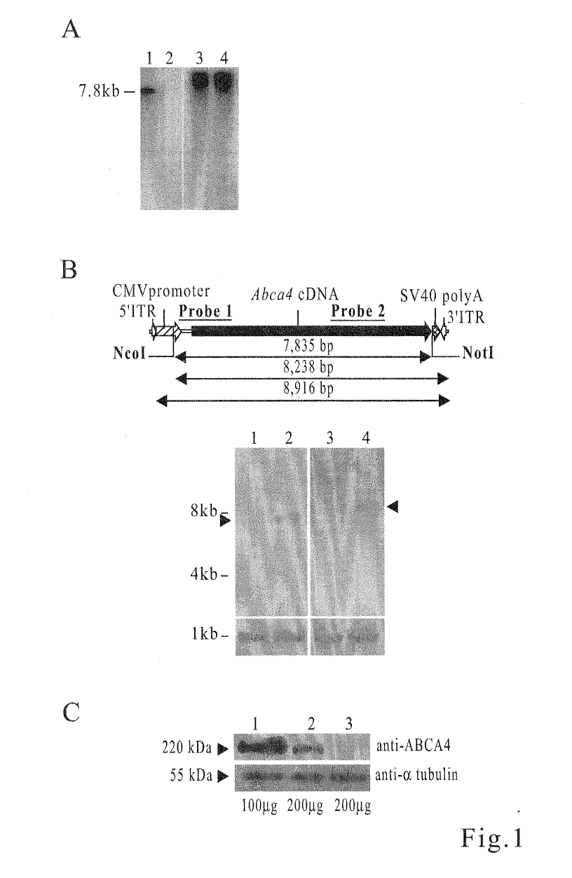

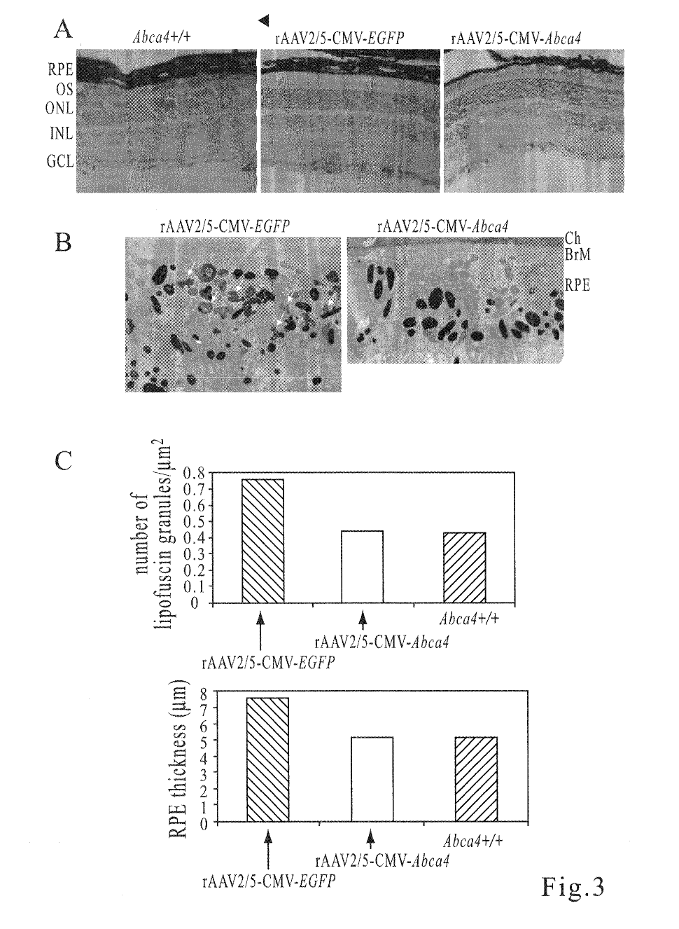

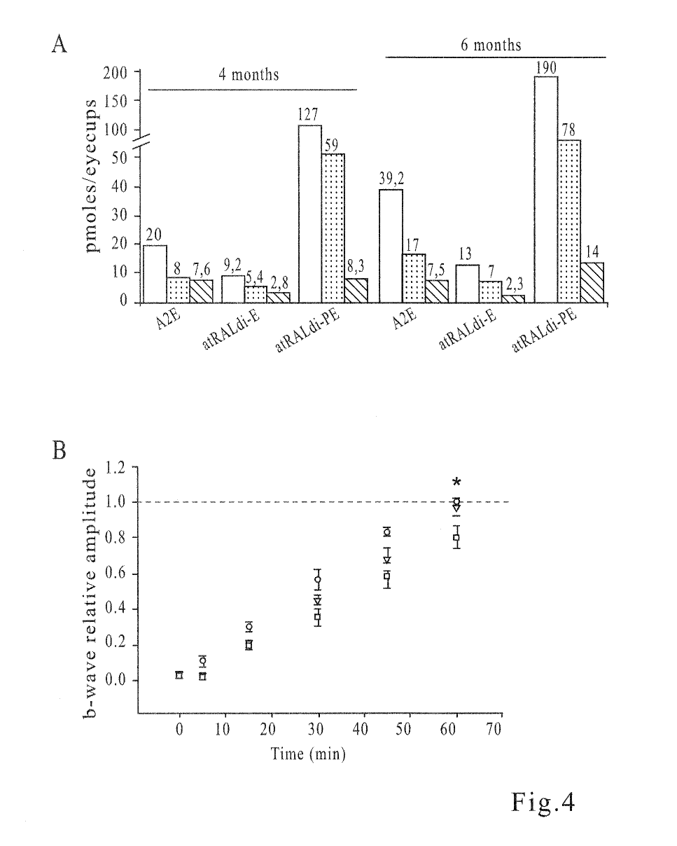

All procedures on animals were performed in accordance with institutional guidelines for animal research. Pigmented (Weng, J., et al., G. H. 1999. Insights into the function of Rim protein in photoreceptors and etiology of Stargardt's disease from the phenotype in abcr knockout mice. Cell 98:13-23) and albino Abca4−/− (Radu, R. A., et al., G. H. 2004. Light exposure stimulates formation of A2E oxiranes in a mouse model of Stargardt's macular degeneration. Proc Natl Acad Sci USA 101:5928-5933) mice generated through successive crosses and backcrosses with Balb/c mice [homozygous for Rpe65 Leu450(44)], Shaker 1 mice [carrying the 4626SB allele, an effective null mutation on a C57BL/6HN_{SD} background (Gibson, F. et al., S. D. 1995. A type VII myosin encoded by the mouse deafness gene shaker-1. Nature 374:62-64)] and wild type C57/BL6 and Balb/c mice (Harlan Italy) were used. Either subretinal or intramuscular injections were performed. Subretinal vector administration was performed in 1-month old Abca4−/− mice as described (Liang, F. Q. et al., J. 2000. Intraocular delivery of recombinant virus. Methods In Molecular Medicine 47:125-139). Subretinal administration and intramuscular injections were supplemented with 40 μM of proteasome inhibitors (LnLL, Sigma Aldrich) to increase rAAV transduction for the experiments depicted in FIGS. 1B and 3 (Grieger, J. C., and Samulski, R. J. 2005. Packaging capacity of adeno-associated virus serotypes: impact of larger genomes on infectivity and postentry steps. J Virol 79:9933-9944). For the in vivo experiments aimed at assessing AAV-mediated morphological and functional rescue (FIGS. 3 and 4) proteasome inhibitors were not used. Before vector administration, mice were anesthetized with an intraperitoneal injection of avertin at 2 ml/100 g body weight (Papaioannou, V. E., and Fox, J. G. 1993. Efficacy of tribromoethanol anesthesia in mice. Lab Anim Sci 43:189-192). Then, mice were injected with 2 μl of rAAV2/5-CMV-Abca4 (1.2×10⁹ GC) in the right eye. The same dose of rAAV2/5-CMV-EGFP was delivered to the left eye, as negative control. Intramuscular (IM) injections were performed in the right gastrocnemius of C57/BL6 mice with 150 μl of rAAV2/5-CMV-Abca4 (9×10¹⁰ GC).

Statistical Analyses

Data are presented as average ± standard errors. Student t-test analysis, ANOVA and a multiple comparison test with a Bonferroni adjustment for multiplicity were used to determine statistical significance where indicated.

Southern Blot Analyses of rAAV Vector DNA

DNA was extracted from 2.5×10¹⁰ viral particles (measured as genome copies). To digest unpackaged genomes, the vector solution was incubated with 11 μl of DNase (Roche) in a total volume of 250 μl, containing 50 mM Tris pH7.5 and 1 mM MgCl₂ for 1 hr at 37° C. The DNase was then inactivated with 50 mM EDTA, followed by incubation at 50° C. for 45 min with proteinase K and 2.5% N-lauryl-sarcosil solution to lyse the capsids. The DNA was extracted twice with phenol-chloroform and precipitated with 2 volumes of ethanol and 10% Sodium Acetate 3M. Alcaline agarose gel electrophoresis was performed as previously described (Sambrook, J.a.D.W.R. 2001. Molecular cloning: a laboratory manual. Cold Spring Harbor, N.Y.: Cold Spring Harbor Laboratory Press). Markers were produced by double-digestion of the pZac2.1-CMV-Abca4 with Ncol and Notl, to produce a band of 7,835 bp. Probe 2 was used to identify rAAV2/5-CMV-Abca4 (FIG. 1B, top panel) while to identify all the other rAAV vector DNA a probe specific for the polyA sequence was used. All probe sequences are available on request.

Southern Blot Analysis of Muscle Genomic DNA Following Transduction with rAAV

DNA was isolated from mouse gastrocnemius 21 days after IM injections by the Hirt extraction method (Yang, G. S., et al. 2002. Virus-mediated transduction of murine retina with adeno-associated virus: effects of viral capsid and genome size. J Virol 76:7651-7660; Hirt, B. 1967. Selective extraction of polyoma DNA from infected mouse cell cultures. J Mol Biol 26:365-369). The DNA (30 μg) was digested with Ncol and Notl or Ncol alone, separated on a 0.8% agarose gel and detected with probes 1 and 2 (FIG. 1B, top panel) or with a probe specific for PDE6B gene (used as loading control) radiolabeled using the Rediprime™ II Random prime labeling system (Amersham) and α-32-CTP according to manufacturer instructions.

rAAV Infection of Cos Cells

Cos cells were plated in 6-well plates to a concentration of 3×10⁵ cell/well. Forty-four hrs later, the cells were incubated with 10⁵ GC/cell of rAAV2/5-CMV-EGFP or rAAV2/5-CMV-Abca4 in serum free DMEM with 10 μM proteasome inhibitors. Forty-eight hrs later the cells were harvested by scraping for Western blot analyses.

Analysis of ABCA4 Expression by Western Blot

Western blot was performed on retinas and on Cos cells infected with rAAV. Retinas were harvested as described (Auricchio, A., et al. J. 2002. Pharmacological regulation of protein expression from adeno-associated viral vectors in the eye. Mol Ther 6:238). Samples were lysed in SIE buffer [250 mM sucrose, 3 mM imidazoles (pH7.4), 1% ethanol and 1% NP-40] on ice for 30 min, proteins were denatured by heating at 37° C. for 30 min in sample buffer with 8M urea and separated by 6% SDS-PAGE. After blotting, specific proteins were labeled using anti-ABCA4 (Santa Cruz Biotechnology), anti-α tubulin (Sigma), and anti-RGR (mcDE5, RGR was used as loading control) antibodies.

Photo-Affinity Labeling Assay on Infected Cos Cells and Retinas

Protein extraction from Cos membranes was performed forty-eight hrs post-infection with rAAV. Cells were harvested in hypotonic buffer [10 mM Tris-HCl (pH 7.4) and 0.5 mM EDTA]. After 1 h at 4° C., the samples were passed through a 28-G needle to disrupt the cells and centrifuged for 1 h at 16,000×g. The resulting membrane pellet was dissolved in the resuspension buffer [25 mM HEPES (pH 7.5), 150 mM NaCl and 5 mM MgCl₂].

Proteins were extracted from rod outer segments by vortexing retinas in 100 μl of 45% sucrose, 20 mM Tris-HCl (pH 7.4), 1 mM EDTA, 2 mM MgCl₂, 20 μM leupeptin, and 2 mM PMSF (21). Then, retinas were centrifuged for 10 min at 4,000×g, the supernatants were collected, diluted with an equal volume of 150 mM NaCl, 20 mM Tris-HCL (pH 7.4), 1 mM EDTA and 2 mM MgCl₂, and recentrifuged for 1 h at 16,000×g. The outer segment pellet was dissolved in 30 μl of resuspension buffer.

For photo-affinity labelling assay, protein extracts from Cos membranes or rod outer segments were incubated at RT with 4 μM 8-Azido-[α-³²P]-ATP (Affinity Labeling Technologies Inc.) for 1 min under ultraviolet light (320 nm) at a distance of 10 cm (Sun, H., Smallwood, P. M., and Nathans, J. 2000. Biochemical defects in ABCR protein variants associated with human retinopathies. Nat Genet 26:242-246). Samples were then mixed with SDS-PAGE sample buffer without heating and the proteins were resolved by SDS-PAGE. 8-Azido-[α-³²P]-ATP labeled proteins were detected with a PhosphorImager (Amersham) by autoradiography.

Extraction and HPLC Analysis of RPE Lipofuscin Pigments

HPLC analysis was performed on eyecups from 4-month old albino and 6-month old pigmented Abca4−/− mice injected with rAAV2/5-CMV-Abca4 in one eye and rAAV2/5-CMV-EGFP in the contralateral eye. Eyecups from age matched Abca4+/+ and Balb/c mice were used as control. Posterior eyecups of dark adapted mice were pooled (4 eyecups per sample), homogenized and extracted three times in chloroform/methanol (1:1) (Kim, S. R., et al. 2004. Rpe65 Leu450Met variant is associated with reduced levels of the retinal pigment epithelium lipofuscin fluorophores A2E and iso-A2E. Proc Natl Acad Sci USA 101:11668-11672). After centrifugation (1,000×g for 2 min.), the organic extract was filtered through cotton and a reversed phase (C8 Sep-Pak, Millipore) cartridge with 0.1% TFA in methanol. The extract was subsequently concentrated by evaporation of solvent under argon gas, redissolved in 50% methanolic chloroform (1 or 2 eyes/10 μL solvent) and analyzed by reverse-phase HPLC using an Alliance System (Waters) equipped with 2695 Separation Module, 2996 Photodiode Array Detector and a 2475 Multi λ Fluorescence Detector. For chromatographic separation, an analytical scale Atlantis® dC18 (3 μm, 4.6×150 mm, Waters) column was utilized with an acetonitrile and water gradient and 0.1% trifluoroacetic acid (90-100%, 0-10 min; 100% acetonitrile, 10-20 min; monitoring at 430 nm; 10 μL injection volume). Extraction and injection for HPLC were performed under dim red light. Integrated peak areas were determined using Empower® software, and picomolar concentrations per eyecup were calculated by reference to an external standard of synthesized compound and by normalizing to the ratio of the HPLC injection volume versus total extract volume. The structures of synthesized standards of A2E, atRALdi-E and atRALdi-PE have been confirmed (Fishkin, N. E.,et al. 2005. Isolation and characterization of a retinal pigment epithelial cell fluorophore: an all-trans-retinal dimer conjugate. Proc Natl Acad Sci USA 102:7091-7096; Sakai, N., et al. J. A. C. 1996. J. Am. Chem. Soc.:1559-1560; Fishkin, N., et al. 2004. Absolute configurational determination of an all-trans-retinal dimer isolated from photoreceptor outer segments. Chirality 16:637-641).

Electrophysiological Recordings

Electrophysiological analysis (ERG) was performed in 4-month old albino Abca4−/− and wild type, age-matched Balb/c mice. Flash ERG was evoked by 10-ms flashes of light generated through a Ganzfeld stimulator (Lace). The electrophysiological signals were recorded through gold-plated electrodes inserted under the lower eyelids in contact with the cornea previously anesthetized with ossibuprocaine (Novartis Pharma). The electrode in each eye was referenced to a needle electrode inserted subcutaneously at the level of corresponding frontal region. The different electrodes were connected to a two-channel amplifier. After 180 min of dark adaptation, mice were anesthetized and loosely mounted in a stereotaxic apparatus under dim red light with the body temperature maintained at 37.5° C. Mice were then exposed to a constant light, the intensity of which was set at 300 cd/m2 for 80 sec (pre-adapting light, bleaching condition). Recovery of b-wave was monitored at fixed intervals after pre-adapting light (0, 5, 15, 30, 45, 60 min). The amplitude of b-wave in response to a flash of 1 cd m⁻² s⁻¹ after the pre-adapting light was measured and expressed as a relative value with respect to that measured before the pre-adapting light.

Electron Microscopic, Histological Analyses and Immunohistochemistry

Mice were perfused through the heart with 2% paraformaldehyde and 1% glutaraidehyde in PBS (pH7.4). Then the eyeballs were removed and fixed overnight in 0.1M Sodium Cacodylate buffer (pH7.4) containing 2% paraformaldehyde and 2% glutaraldehyde. The fixed eyeballs were cut so that the lens and vitreous could be removed leaving the eyecup. The eyecups were treated with 1% osmium tetroxide and stained with 1% aqueous uranyl acetate. The specimens were then dehydrated and embedded in Epon-812. Thin sections from the temporal side of each eye, which corresponds to the injected side, were prepared on an Ultracut microtome (Leica). EM images were acquired from thin sections under a FEI Philips Tecnai-12 electron microscope (Philips) using an ULTRA VIEW CCD digital camera. Micrographs were obtained at 6,000× magnification. Quantitative analysis of numbers of lipofuscin granules was made by counting on three different optical fields for each eye the smaller structures of variable density representing lipofuscin granules distinct from the large oval structures of high electron density representing melanosomes. RPE thickness measurements were done in 20 different places per specimen (10 measurements across the nuclear area where the cell is thicker ad 10 across cell-to-cell border where the cell is thinner). Then, the counts were averaged.

For histological analysis mouse eyecups were harvested, fixed by immersion in 4% paraformaldehyde and embedded in OCT (kaltek). For each eye serial sections (11 μm-thick) were cut along the horizontal meridian and distributed on 10 slides so that each slide contained representative sections of the whole eye at different levels. The sections were stained with hematoxylin and eosin (Sigma-Aldrich) and retinal histology was analyzed by light microscopy. For the ABCA4 staining, the tissue sections were incubated for 1 h with blocking solution [1× PBS, 0.5% Tween-20, 0.1% bovine serum albumin) and 10% fetal bovine serum (GIBCO BRL-Invitrogen) before incubation overnight with the Rim 3F4 antibody (a kind gift of Robert S. Molday, University of British Columbia, Vancouver, British Columbia, Canada). After washing, sections were incubated for 1 h with secondary anti'-mouse IgG conjugated to HRP (Vector laboratory) followed by 30′ DAB staining (Vector laboratory). The counterstaining was performed for 1 min with Hematoxilin (Sigma-Aldrich). Stained sections were mounted with Eukitt (Kaltek).

Results

rAAV2/5 Administration in a Mouse Model of rSTGD Significantly Improves Retinal Morphology and Function

Based on the results above, we tested the efficacy of AAV2/5-mediated retinal gene transfer in a murine model of rSTGD. Targeted disruption of the Abca4 locus in pigmented (Weng, J., et al., G. H. 1999. Insights into the function of Rim protein in photoreceptors and etiology of Stargardt's disease from the phenotype in abcr knockout mice. Cell 98:13-23) and albino (Radu, R. A., et al. G. H. 2004. Light exposure stimulates formation of A2E oxiranes in a mouse model of Stargardt's macular degeneration. Proc Natl Acad Sci USA 101:5928-5933) mice (Abca4−/−) results in a phenotype that recapitulates some rSTGD characteristics: accumulation of lipofuscin in the RPE, thicker RPE cells, slow photoreceptor degeneration and delayed dark adaptation (Weng, J., et al., G. H. 1999. Insights into the function of Rim protein in photoreceptors and etiology of Stargardt's disease from the phenotype in abcr knockout mice. Cell 98:13-23; Radu, R. A., et al. G. H. 2004. Light exposure stimulates formation of A2E oxiranes in a mouse model of Stargardt's macular degeneration. Proc Natl Acad Sci USA 101:5928-5933; Mata, N. L., et al. G. H. 2001. Delayed dark-adaptation and lipofuscin accumulation in abcr± mice: implications for involvement of ABCR in age-related macular degeneration. Invest Ophthalmol Vis Sci 42:1685-1690). To test whether rAAV2/5-mediated gene delivery results in correction of the Abca4−/− mutant phenotype, 1 month-old mice were injected subretinally with 2 μl of rAAV2/5-CMV-Abca4 (corresponding to 1.2×10⁹ GC) in one eye and with the same dose of rAAV2/5-CMV-EGFP in the contralateral eye. The impact of gene transfer on Abca4−/− retinas was evaluated 3 months later (age of the animals: 4 months) unless otherwise noted. We initially analyzed recombinant ABCA4 expression by immunohistochemistry on retinal sections and found that it properly localizes to photoreceptor outer segments (FIG. 3A) as the endogenous ABCA4 does and as expected by the reported rAAV2/5 tropism.

We then evaluated the impact of rAAV2/5-mediated gene transfer on Abca4−/− RPE abnormalities such as presence of lipofuscin granules and thicker RPE. Electron microscopy analysis of RPE cells located in the region of injection revealed a reduced number of lipofuscin granules and decreased RPE thickness (both similar to that seen in Abca4+/+ RPE) in the Abca4−/− retinas treated with rAAV2/5-CMV-Abca4 when compared to those treated with rAAV2/5-CMV-EGFP (FIGS. 3B and C). This suggests that rAAV2/5-mediated Abca4 gene transfer ameliorates the RPE ultrastructural abnormalities associated with the Abca4−/− phenotype.

Consistent with a role for ABCA4 in the transport of N-retinylidene-phosphatidylethanolamine across photoreceptor disk membranes (Sun, H., et al. J. 1999. Retinal stimulates ATP hydrolysis by purified and reconstituted ABCR, the photoreceptor-specific ATP-binding cassette transporter responsible for Stargardt disease. J Biol Chem 274:8269-8281; Beharry, S., et al. 2004. N-retinylidene-phosphatidylethanolamine is the preferred retinoid substrate for the photoreceptor-specific ABC transporter ABCA4 (ABCR). J Biol Chem 279:53972-53979), the lipofuscin granules present in the RPE of Abca4−/− mice contain the bisretinoid fluorophores A2E, all-trans-retinal-dimer-ethanolamine (atRALdi-E) and all-trans-retinal-dimer-phosphatidylethanolamine (atRALdi-PE) (Fishkin, N. E.,et al. 2005. Isolation and characterization of a retinal pigment epithelial cell fluorophore: an all-trans-retinal dimer conjugate. Proc Natl Acad Sci USA 102:7091-7096). The levels of the fluorophores A2E, atRALdi-E and atRALdi-PE were significantly reduced in both albino (age: 4 months) and pigmented (age: 6 months) Abca4−/− retinas treated with rAAV2/5-CMV-Abca4, when compared with the EGFP-treated contralateral eyes (FIG. 4A). In addition, the ability of Abca4−/− photoreceptors to recover from light desensitization was significantly improved in the retinas treated with the therapeutic vector when compared to control EGFP-treated retinas (FIG. 4B). Hematoxilin and eosin staining of retinal sections did not reveal any inflammatory infiltrate, or a reduction in the outer nuclear layer thickness in either Abca4 or EGFP-treated eyes.

I claim:

1. A method for correcting retinal abnormalities and/or retinal function in a subject affected by a disease associated with mutations in ABCA4 gene, said method comprising the following steps:
1) providing a recombinant adeno-associated viral (AAV) vector with AAV5 capsid, said vector carrying an expression cassette which contains a nucleic acid molecule encoding a functional ABCR protein, wherein said nucleic acid molecule is operably linked to regulatory control elements that direct the transcription and translation thereof;
2) transducing photoreceptor cells with said recombinant AAV vector, whereby the expression of the ABCR protein is induced in said cells.

2. The method according to claim 1, wherein said subject is human.

3. The method according to claim 1, wherein said disease is selected from recessive Stargardt's disease, cone-rod dystrophy, retinitis pigmentosa and age-related macular degeneration (AMD).

4. The method according to claim 1, wherein said vector with AAV5 capsid is able to package up to 9 kb of nucleic acid.

5. The method according to claim 4, wherein said vector is AAV2/5.

6. The method according to claim 1, wherein said recombinant adeno-associated viral (AAV) vector with AAV5 capsid carries an expression cassette in which a coding sequence of ABCA4 is functionally linked to a promoter sequence able to regulate its expression in mammalian retinal cells.

7. The method according to claim 6, wherein said coding sequence of ABCA4 consists of SEQ ID NO:1, or a sequence encoding the same amino acid sequence as SEQ ID NO:1.

8. The method according to claim 6, wherein said promoter sequence is selected from SEQ ID NO:2, SEQ ID NO:3, SEQ ID NO:4 and SEQ ID NO:5, fragments or variants thereof which retain a transcription promoter activity.

9. The method according to claim 1, wherein transduction of photoreceptor cells is effected by subretinal administration of said vector or a pharmaceutical preparation thereof.

10. A recombinant adeno-associated viral (AAV) vector with AAV5 capsid carrying an expression cassette in which a coding sequence of ABCA4 is functionally linked to a promoter sequence able to regulate its expression in mammalian retinal cells.

11. The vector according to claim 10, wherein said vector is AAV2/5 serotype.

12. A pharmaceutical preparation containing an AAV vector as defined in claim 10, in a form suitable for ocular administration.

13. The pharmaceutical composition according to claim 12, which is in the form of an injectable solution or suspension, eye lotion or ophthalmic ointment.
